(19) United States
(12) Patent Application Publication
(10) Pub. No.: US 2003/0124654 A1
(43) Pub. Date: Jul. 3, 2003

(54) Method and device for the rapid clinical diagnosis of hepatitis C virus (HCV) infection in biological samples

(75) Inventors: Vijay Sharma, Mumbai (IN); Venkata Ramana Kondiboyina, Mumbai (IN)

Correspondence Address: LACKENBACH SIEGEL, ONE CHASE ROAD, SCARSDALE, NY, 10583, US

(73) Assignee: Reliance Life Sciences Private Limited, Mumbai (IN)

(21) Appl. No.: 10/037,990

(22) Filed: Jan. 3, 2002

(51) Int. Cl. C12Q 1/70; C12Q 1/68; C07H 21/02; C07H 21/04; C12P 21/06; C12P 19/34; A61K 39/29

(52) U.S. Cl. 435/69.1; 435/5; 435/6; 536/24.33; 536/23.72; 536/23.1; 435/91.33; 424/225.1

(57) ABSTRACT

There is provided a method and kit for rapid clinical diagnosis of HCV in which the amplimers are transcripts of a polyprotein gene of HCV. The amplicons are hybridized to a specific oligonucleotide probe, which allows the amplicons to be detected.

### BACKGROUND OF THE INVENTION

1. Field of Invention

The present invention relates to methods and devices for the diagnosis of infections. In particular, the present invention relates to methods and kits for detection of Hepatitis C Virus Viremia (HCV).

2. Description of the Prior Art

Hepatitis C Virus (HCV) is a blood borne pathogens that is present in infected human blood and can infect and cause disease in persons who are exposed to blood containing the pathogen. HCV constitute a highly variable genus within the Flaviviridae with closest homology to the hepatitis G and GB virus and Pestiviruses. The positive stranded RNA genome encodes a polyprotein, which is co and post translationally cleaved into at least 9 (nine) proteins : Core, E1, and E2 (the structural proteins) NS2, NS3, NS4A,NS4B, NS5A and NS5B (the non-structural (NS) proteins) HCV is a single-stranded, positive sense RNA virus with a genome of approximately 10,000 nucleotides coding for 3000 amino acids.

HCV has become the most significant cause of chronic liver disease of infectious etiology. Hepatitis C has infected over 200 million worldwide. The recognition that HCV can be transmitted perinatally or through transfusions and transplantation warrants particular attention. Although HCV can progress to irreversible liver damage, acute infection is often asymptomatic. Complications from this disease are already the leading cause of liver transplants.

Accurate diagnosis is essential to identify the presence of the virus in apparently symptom free carriers. Nucleic acid detection strategies allow the identification of the virus at extremely low levels. The use of HCV identification has proven valuable because the presence of RNA is an indicator of replicating HCV, whereas the presence of antibodies is not.

Several recent studies have indicated that the level of HCV viremia correlates with the clinical stage of disease; patients with advanced stages of liver disease such as severe chronic active hepatitis, cirrhosis and end stage liver disease had higher serum levels of HCV RNA than patients with mild HCV infections. Moreover, the HCV viremia titer may predict a subsequent response to antiviral therapy.

Early and rapid diagnosis of HCV infection is of great importance. Yet, conventional methods for detection of HCV from blood are inaccurate and/or slow. The presence of anti-HCV antibodies in patients infected with HCV has led to the development of the immunoserological tests that are specific for these antibodies. Implementation of these tests already has reduced the incidence of post-transfusion hepatitis worldwide. Additional supplemental testing has been performed using the RIBA (recombinant immunoblot assay) to further evaluate samples that are repeatedly reactive in the antibody screening assay.

Recent evaluations have shown that interpretation of these immunological tests is difficult since 25-90% of the samples that are repeatedly reactive in the screening assay are negative upon supplemental evaluation with the RIBA assay. Immunoserological testing is a measure of current and/or prior exposure to HCV infection, but cannot discriminate between the two. At present, an immunolological assay for direct detection of HCV antigen is unavailable. Furthermore, in cases of acute HCV infection, individuals may fail to produce antibody to HCV, making diagnosis of current HCV infection impossible using Immunoserological techniques. Alternatively, detection of HCV RNA by Polymerase Chain Reaction (PCR) provides evidence for ongoing infection. Using PCR, it is possible to detect HCV viremia prior to Immunoserological sero-conversion. This procedure is also valuable in detecting HCV RNA in immunocompromised patients.

The following requirements need to be fulfilled for an optimal assay for HCV diagnosis.

High sensitivity and specificity;

Rapid results; and

High reproducibility.

Kits for detection of HCV are commercially available. One such kit is produced by Hoffmann-La Roche and sold under the tradename Amplicor. This kit makes use of amplification by Polymerase Chain Reaction (PCR) to create amplicons specific to HCV followed by Enzyme Linked Immunosorbent Assay (ELISA) to detect the amplicons.

In light of the foregoing, there is a need for a more sensitive and specific detection protocol for clinical samples. The inventors of the present invention have been successful in developing a kit and a method for detecting HCV in a more sensitive, specific and rapid manner. The present invention obviates the problems associated with the conventional kits.

Several terms used in the invention are defined as follows.

The term “primer” refers to a synthetic oligonucleotide sequence synthesized for annealing to a specific nucleotide sequence of interest. The primer initiates DNA synthesis to occur using thermostable DNA dependent DNA polymerase. Selecting the proper primer is one of the most important steps in designing a PCR kit. The primer set must hybridize to the target sequence with little or no hybridization to other sequences that are also present in the sample.

The term “probe” refers to a synthetic oligonucleotide sequence which lies internal to the + strand of the amplified product resulting from a PCR reaction.

The term “hybridization” refers to annealing of nucleotide sequences to each other under optimal conditions. Typically, a nucleotide A binds to nucleotide T and nucleotide G binds to nucleotide C.

The term “biological samples” refers to the samples selected from serum, plasma, and combinations thereof. When selected plasma will preferably include an anticoagulant, such as EDTA/and or ACD.

### SUMMARY OF THE INVENTION

It is an object of the present invention to develop an improved HCV diagnostic kit.

It is an object of the present invention to develop an improved method for rapid clinical diagnosis of HCV infection.

It is still an object of the invention is to develop a method using specific primer sets and detection probe having higher sensitivity and specificity as compared to the conventional assays.

These and other objects of the present invention are substantially achieved by a method and kit for rapid clinical diagnosis of HCV in which the amplimers are transcripts of a polyprotein gene of HCV. The amplicons are hybridized to a specific oligonucleotide probe, which allows the amplicons to be detected.

### DESCRIPTION OF THE INVENTION

Selecting the target DNA sequence polyprotein gene for HCV depends upon identification of regions within the HCV genome that show maximum sequence conservation among all the serotypes of HCV.

The present invention has primers complementary to sequences flanking a segment polyprotein gene to be amplified. The primers of the present invention are selected on their ability to specifically recognize the polyprotein gene with a low mutation frequency.

The primers of the present invention are so designed to avoid hairpin loop structure formation. In addition, the selected primers have been subjected to a gene bank search to identify homologies and percent similarities to the target of interest. The Blast results are as follows:

For nucleic acid SEQ. ID. NO. 1, SEQ. ID. NO. 2, and SEQ. ID. NO. 3, the Accession No. AY-051292 yielded 100% homology.

A diagnostic kit for detection of HCV in biological samples according to the present invention includes four components. The first component is the extraction of nucleic acid (RNA) from the biological sample. The second component is the reverse transcription of the extracted nucleic acid (RNA) with the reverse strand primer. The third component is amplification component, which is used to amplify the target sequence. The fourth component is the detection component, which is used to detect the amplicons produced by the amplification component.

The amplification component amplifies the target sequence via PCR and, therefore, will include a pair of amplification oligonucleotide primers, a RNA-dependent DNA polymerase; a DNA-dependent RNA polymerase, and deoxyribonucleoside triphosphates. The primers are labeled at their 5′ end. Labels are preferable selected from the group consisting of biotin, digoxigenin, radioactive labels (³²P). A more preferable label is fluorescein.

The first oligonucleotide primer for use in reverse transcription and amplification component has the following nucleic acid sequence (SEQ. ID. NO. 1):

5′-gcagaaagcgtctagccatggcgt-3′

SEQ. ID. 1 contains twenty four (24) continuous bases selected from the polyprotein gene sense strand. The Nucleotide Sequence position of SEQ. ID. NO. 1 is 56-79. SEQ. ID. NO. 1 is preferably present in the amplification component in an amount of about 1 μL to about 10 μL in a concentration of 10-100 pM.

The second oligonucleotide primer for use in the amplification component has the following nucleic acid sequence (SEQ. ID. NO. 2):

5′-ctcgcaagcaccctatcaggcagt-3′

SEQ. ID. 2 contains twenty four (24) continuous bases selected from the polyprotein gene antisense strand. The Nucleotide Sequence position of SEQ. ID. NO. 2 is 276-299. SEQ. ID. NO. 2 is preferably present in the amplification component in an amount of about 1 μL to about 10 μL in a concentration of 10-100 pM.

The annealing temperature of primer pair is generally calculated as 5° C. lower than the estimated melting temperature. The annealing temperature for primers that are less than 20 bases is calculated using the following formula: [4(G+C)+2 (A+T)]−5° C. Ideally the annealing temperature of each primer should match and be within the 55° C. and 75° C. range. If the annealing temperature difference between the two primers is high, the lower annealing temperature can be increased adding to the length of that primer at either the 3′ end (this can also keep the size of the amplified locus constant) or the 5′ end. The annealing temperature for the primers of the present invention is about 60° C.

The RNA-dependent DNA polymerase may be any suitable reverse transcriptase. Preferably, the reverse transcriptase is derived from Avian Myeloblastosis Virus (AMV). The said polymerase is preferably present in an amount of about 5 Units to about 10 Units.

The reverse transcription is set up using reverse transcriptase buffer comprising of 50 mM Tris.Hcl (pH 8.3), 6.0 mM MgCl₂, 40 mM KCl, 4.0 mM dithiothreitol, 5-10 units of AMV reverse transcriptase, dNTP's in a concentration of 100-200 μM and is incubated at about 37° C. to about 42° C. for 30 min.

The DNA-dependent DNA polymerase may be any suitable polymerase. Preferably, the polymerase is derived from Thermus aquaticus (Taq) bacteria. The polymerase is preferably present in the amplification component in an amount of about 1 Unit to about 2.5 Units.

The deoxyribonucleoside triphosphates (dNTPs) useful in the present invention include: dATP, dCTP, 5MedCTP, dGTP, dITP, TTP, and dUTP. Preferably, the dNTPs are selected from dATP, dCTP, dGTP, dTTP, and combinations thereof. Preferably, each dNTP is present in the amplification component in an amount of about 100 μM to about 200 μM.

The amplification component may contain any other suitable additional ingredient and/or component, such as an amplification buffer. For example, suitable 10× amplification buffers for use in the present invention include 100 mM Tris HCl (pH 8.3), 500 mM KCl, and MgCl₂.

A preferred amplification component includes:

(1) an amplification buffer having 10 mM Tris HCl (pH 8.3) and 500 mM KCl;

(2) about 100 to about 200 μM each of dATP, dCTP, dTTP and dGTP;

(3) sterile distilled water(nuclease free);

(4) about 1 unit to about 2.5 units of thermostable DNA-dependent DNA polymerase;

(5) about 10 to about 100 pM of the first oligonucleotide primer (SEQ. ID. NO. 1);

(6) about 10 to about 100 pM of the second oligonucleotide primer (SEQ. ID. NO. 2);

(7) about 1.5 to about 2.5 mM MgCl₂; and

(8) the template to be amplified.

Preferably, the volume of the amplification buffer is about 25 to about 50 μL and the volume of the extracted sample is about 25 to about 50 μL. The final volume of the amplification component is about 50 to about 100 μL.

The detection component detects the amplified target sequence via ELISA and, therefore, will include a oligonucleotide probe immobilized upon a solid medium, a conjugate that is adapted to bind to a label present on the amplicons, and a complex that changes color in the presence of the conjugate.

The oligonucleotide probe has the following a nucleic acid sequence (SEQ. ID. NO. 3):

5′-gtcgtgcagccctccaggaccc-3′

The Nucleotide Sequence position of SEQ. ID. NO. 3 is 90-110 and contains twenty one (21) continuous bases. SEQ. ID. NO. 3 is a region internal to the amplimers created using the primers of SEQ. ID. NO. 1 and SEQ. ID. NO. 2.

SEQ. ID. NO. 3 is specifically designed for capturing the amplification product. The oligonucleotide probe is preferably immobilized on a solid medium, such as a microwell plate. For example, the oligonucleotide probe may be labeled with biotin, which is substantially irreversibly bound to streptavidin coating the microwell plate.

The oligonucleotide probe is preferably present in the detection component in an amount of about 10 μL to about 100 μL in a concentration of 10-100 pM.

The enzyme on the conjugate may be any enzyme, depending only upon the selected substrate. For example, the preferred enzyme for the present invention is horse raddish peroxidase. However, other enzymes such as alkaline phosphatase may be used.

The substrate changes color in the presence of the enzyme conjugate to visibly show the presence of the amplimer bound to the oligonucleotide probe. Thus, a change in the color detection solution positively indicates the presence of the amplimer and, by extension, the presence of HCV in the original biological sample. The selection of the substrate is dependent upon the selected enzyme on the conjugate. A preferred enzyme and substrate combination for use in the present invention is hydrogen peroxide (H₂0₂) and 3,3′,5,5′-Tetra methyl benzidine Dihydrochloride(TMB), present in an amount of 100 microlitres, which is oxidized by H₂0₂ in the presence of peroxidase and, thus, the detection solution changes from colorless to blue. Other suitable enzyme and substrate combinations are as follows:

Alkaline Phosphatase and 5-Bromo-4-Chloro-3-Indolyl Phosphate (BCIP)

Alkaline Phosphatase and Fast Red RC

Alkaline Phosphatase and Naphthol AS-TR Phosphate

Alkaline Phosphatase and Nitro BlueTetrazolium (NBT)

Alkaline Phosphatase and p-Nitrophenyl Phosphate (pNPP)

Peroxidase and 3-Amino-9-Ethylcarbazole (AEC)

Peroxidase and 5-Aminosalicyclic acid (5AS)

Peroxidase and 2,2′-Azino-bis(3-Ethylbenzthiazoline-6-Sulfonic acid)

Peroxidase and 4-Chloro-1-Naphthol (4CIN)

Peroxidase and 3-3′DiaminobenzidineTetrahydro-chloride (DAB)

Peroxidase and o-Dianisidine

Peroxidase and o-Phenylenediamine Freebase (OPD)

A method for rapid clinical diagnosis HCV according to the present invention uses the first primer (SEQ. ID. NO. 1) and the second primer (SEQ. ID. NO. 2) in an amplification step, and the oligonucleotide probe (SEQ. ID. NO. 3) in a detection step.

In accordance with a second aspect of this invention, a method according to the present invention includes the steps of sample extraction, reverse transcription, amplification (preferably by PCR), and detection by enzyme immunoassay (preferably ELISA).

HCV nucleic acid is extracted from a biological sample, preferably using chaotropic agents such as urea, diethylamine, guanidium hydrochloride, potassium iodide, sodium dodecyl sulphate (SDS), Formamide and combinations thereof. Any suitable and/or known technique for extraction of nucleic acid may be used.

The extracted specimen is subject to reverse transcription and is then added to a mixture. The mixture contains the reverse strand primer of the present invention (SEQ. ID. NO. 2), deoxyribonucleoside triphosphates, and AMV reverse transcriptase (RNA dependent DNA polymerase). The appropriate reverse transcriptase buffer comprises of 50 mM Tris.Hcl (pH 8.3), 6.0 mM MgCl₂, 40 mM KCl, 4.0 mM dithiothreitol, 5-10 Units of AMV reverse transcriptase, dNTP's in a concentration of 100-200 μM and is incubated at about 37° C. to about 42° C. for 30 min.

The resulting complimentary DNA strand is then added to an amplification component. As discussed above, the amplification component contains the primers of the present invention (SEQ. ID. NOs. 1 and 2), having a label at their 5′ ends, deoxyribonucleoside triphosphates and Thermus acqauticus (Taq) derived DNA dependent DNA polymerase.

Amplification is accomplished by repeated cycles of DNA denaturation, primer annealing, and extension of the primed DNA sequence by the DNA polymerase in the presence of added purine and pyrimidine bases. In general, each cycle will double the amount of the target DNA sequence. The amplification cycle is repeated until a detectable amount of the DNA sequence has been created. Further details of the PCR method are provided in U.S. Pat. Nos. 4,683,195; 4,683,202; 4,965,188; and 5,075,216, which are incorporated herein by reference in their entirety.

The amplimers are preferably detected using ELISA. Denatured and labeled amplimers are added to a microwell containing the immobilized oligonucleotide probe of the present invention (SEQ. ID. NO. 3) and a hybridization solution, thereby immobilizing the amplimers by hybridization with the oligonucleotide probe. An anti-fluorescein conjugate with a selected enzyme is added to the microwell after excess unbound amplimers are washed away. Finally, a substrate is added to the microwell, which changes color in the presence of the enzyme anti-fluorescein conjugate. Thus, the detection solution changes color if any amplimers are present in the microwell, which denotes a positive result for the presence of HCV in the original biological sample.

This method is rapid, automatable (ELISA type solid phase formats do not require gel electrophoresis) and applicable to large scale screening programs.

The following example illustrates the process according to the invention without limitation.

### EXAMPLE 1

An experiment was conducted to demonstrate the method of present invention. The initial sample taken was plasma EDTA that was pre-determined as positive for the presence of HCV mediated antibodies (IgG, IgM). The sample (0.2 ml) was extracted using RNA isolation system sold by Qiagen Inc., Venlo, The Netherlands, under the tradename QIAamp®.

Reverse transcription was set up using the reverse strand primer in a reverse transcriptase buffer including 50 mM Tris.HCl (pH 8.3), 6.0 mM MgCl₂, 40 mM KCl, 4.0 mM dithiothreitol, 5-10 Units of AMV reverse transcriptase, dNTP's in a concentration of 100-200 μM was added to make the final concentration of about 1.5 mM to about 2.5 mM. The solution/reaction mixture was incubated at 42° C. for about 30 min. The DNA resulting from reverse transcription is thereafter amplified.

The amplification reaction was set up using 25 μL of the above extracted nucleic acid and 25 μL of 10× amplification buffer including 100 mM Tris HCl (pH 8.3), 500 mM KCl, and MgCl₂ was added to make a final concentration of about 1.5 mM to about 2.5 mM. The primers (SEQ. ID. NOs. 1 and 2) were labeled with fluorescein and provided in a concentration of 10-100 pM.

Each dNTP (dATP, dCTP, dGTP, and dTTP) was provided in a concentration of 100-200 μM. AMV polymerase was provided in an amount of about 5-10 units and Taq polymerase was provided in an amount of 1-2.5 units.

This reaction mixture was heated to 94° C. for 10 min followed by 30 amplification cycles. Each amplification cycle included heating the reaction mixture to 94° C. for 30 sec, cooling to 62° C. for 45 sec, and heating to 72° C. for 30 sec. Following the 30 amplification cycles, the final incubation was performed for 10 min at 72° C. The resulting amplimers were denatured using a solution of 0.4M NaOH.

The detection of the fluorescein labeled amplimers was done in the following manner:

50-100 μL of dilution buffer was pipetted into a microwell plate coated with streptavidin, to which was added 1-10 μL of the biotinylated oligonucleotide probe (SEQ. ID. NO. 3) in a concentration of 10-100 pM. The solution was incubated at 37° C. for 30-60 min. Thereafter, the microwell plate was washed with PBS-T wash buffer, and 100 μL of the hybridization buffer was added and incubated for 15-30 min at 37° C. The hybridization buffer included sodium phosphate, sodium thiocyanade and Denhardts solution, which includes Polyvinylpyrollidine (PVP), Ficoll, Bovine Serum Albumin (BSA)

25 μL of denatured amplification product was added to the microwell plate and incubated for 30-60 min at 42° C. Following incubation, the microwell plate was washed 5 times with PBS-T wash buffer. 100 μL of diluted anti-fluorescein-HRP conjugate was added to the washed plate and incubated for 30-45 min at 37° C. Following incubation, the microwell plate was washed 5 times with PBS. 100 μL of TMB was added to the microwell plate and incubated substantially without light at room temperature for 15-30 minutes.

A color change from colorless to blue is observed after incubating for 15 min. 50 μL of a stop solution containing 0.1 N H₂SO₄ was added and the change in color from blue to yellow was noticed and the plate was read at 403 nm in a calorimetric plate reader.

In view of the foregoing descriptions and example, it will become apparent to those of ordinary skill in the art that equivalent modifications thereof may be made without departing from the spirit and scope of this invention. Various features are set forth in the following claims.

We claim:

1. A kit for amplifying HCV nucleic acid comprising:
a first amplification primer having the sequence:
5′-gcagaaagcgtctagccatggcgt-3′ [SEQ. ID. NO. 1]
and a second amplification primer having the sequence:
5′-ctcgcaagcaccctatcaggcagt-3′ [SEQ. ID. NO. 2]

2. The kit according to claim 1, wherein the first amplification primer comprises at least twenty four continuous bases selected from the polyprotein gene sense strand.

3. The kit according to claim 1, wherein the first amplification primer is present in an amount of about 10 to about 100 pM.

4. The kit according to claim 1, wherein the second amplification primer comprises at least twenty four continuous bases selected from the polyprotein gene anti-sense strand.

5. The kit according to claim 1, wherein the second amplification primer is present in an amount of about 10 to about 100 pM.

6. The kit according to claim 1, further comprising RNA dependent DNA Polymerase.

7. The kit according to claim 6, wherein the RNA dependent DNA polymerase is Avian Mycloblastosis Virus present in an amount of about 5 units to about 10 units.

8. The kit according to claim 1, further comprising DNA dependent DNA polymerase.

9. The kit according to claim 8, wherein the DNA dependent DNA polymerase is Taq polymerase present in an amount of about 1 Unit to about 2.5 Units.

10. The kit according to claim 1, further comprising a deoxyribonucleoside triphosphates.

11. The kit according to claim 10, wherein said deoxyribonucleoside triphosphates are selected from the group consisting of dATP, dCTP, 5MedCTP, dGTP, dITP, TTP, dUTP, and combinations thereof.

12. The kit according to claim 11, wherein said deoxyribonucleoside triphosphate is present in an amount of about 100 to about 200 μM.

13. The kit according to claim 1, wherein the first and second amplification primers have a label at their respective 5′ ends.

14. The kit according to claim 13, wherein the label is fluorescein.

15. A kit for detecting an HCV nucleic acid comprising an oligonucleotide probe having the sequence:
5′-gtcgtgcagcctccaggaccc-3′ [SEQ. ID. NO. 3]

16. A kit according to claim 15, wherein the sequence of the oligonucleotide probe is internal to an amplimer resulting from the amplification using SEQ. ID. NO. 1 and SEQ. ID. NO. 2

17. A kit according to claim 15, wherein the oligonucleotide probe has a label at their 5′ end.

18. A kit according to claim 17, wherein the label is biotin.

19. The kit according to claim 15, wherein the oligonucleotide probe is immobilized on a solid medium.

20. The kit according to claim 15, wherein the oligonucleotide probe is present in an amount of about 10 to about 100 pM.

21. The kit according to claim 15, further comprising a conjugate adapted to bind with a label present on the HCV nucleic acid.

22. The kit according to claim 21, wherein the selected label is fluorescein and the conjugate is an anti-fluorescein/horse raddish peroxidase conjugate present in an amount of about 1 Unit to about 4 Units.

23. The kit according to claim 21, further comprising a substrate adapted to change color in the presence of an enzyme on the conjugate.

24. The kit according to claim 23, wherein the detection solution comprises hydrogen peroxide and 3,3′,5,5′-Tetra methyl benzidine Dihydrochloride.

25. The kit according to claim 24, wherein the substrate is present in an amount of about 100 μL.

26. A method for detecting HCV nucleic acid in a biological sample comprising the steps of:
extracting HGV nucleic acid a biological sample;
reverse transcription of the extracted nucleic acid using the reverse strand primer; and
amplifying the HCV nucleic acid using a first primer having the sequence
5′-gcagaaagcgtctagccatggcgt-3′ [SEQ. ID. NO. 1]
and a second primer having the sequence
5′-ctcgcaagcaccctatcaggcagt-3′ [SEQ. ID. NO. 2]
and detecting the HCV nucleic acid using an oligonucleotide probe having the sequence:
5′-gtcgtgcagcctccaggaccc-3′ [SEQ. ID. NO. 3]

27. The method according to claim 26, wherein the biological sample is selected from the group consisting of: serum, plasma, and combinations thereof.

28. The method according to claim 26, wherein the first and second amplification primers have a label at their respective 5′ ends.

29. The method according to claim 28, wherein the label is fluorescein.

30. The method according to claim 26, wherein the step of amplifying the HCV nucleic acid includes:
denaturing the HCV nucleic acid to produce denatured HCV nucleic acid;
annealing the first and second amplification primers to the denatured HCV nucleic acid to produce primed HCV nucleic acid; and
extending the primed HCV nucleic acid using a thermostable DNA dependent DNA polymerase in the presence of a deoxyribonucleoside triphosphate.

31. The method according to claim 30, wherein the DNA dependent DNA polymerase is Taq polymerase present in an amount of about 1 Unit to about 2.5 Units.

32. The method according to claim 30, wherein the deoxyribonucleoside triphosphate is selected from the group consisting of: dATP, dCTP, 5MedCTP, dGTP, dITP, TTP, dUTP, and combinations thereof, and wherein the deoxyribonucleoside triphosphate is present in an amount of about 100 to about 200 μM.

33. The method according to claim 26, wherein the step of detecting the HCV nucleic acid includes:
binding the HCV nucleic acid with the oligonuclotide probe attached to a solid medium to form immobilized HCV nucleic acid;
binding the immobilized HCV nucleic acid with a conjugate; and
adding a substrate that is adapted to change color in the presence of an enzyme on the conjugate,
whereby a change of the color of the substrate indicates the presence of HCV nucleic acid.

34. The method according to claim 33, wherein the HCV nucleic acid is labeled with flourescein, and wherein the detectable marker is an anti-flourescein/horse raddish peroxidase conjugate in an amount of about 1 unit to about 4 units.

35. The method according to claim 33, wherein the substrate compromises hydrogen peroxide and 3,3′,5,5′-Tetra methyl benzidine Dihydrochloride.

36. The method according to claim 33, wherein the substrate is present in an amount of about 100 μL.

37. The method according to claim 33, further comprising the step of reading a change of the color of the substrate with a calorimetric plate reader.


SEQUENCE LISTING (3 sequences)

SEQ ID NO: 1 - DNA, 24 bp, Hepatitis C virus
gcagaaagcgtctagccatggcgt

SEQ ID NO: 2 - DNA, 24 bp, Hepatitis C virus
ctcgcaagcaccctatcaggcagt

SEQ ID NO: 3 - DNA, 22 bp, Hepatitis C virus
gtcgtgcagccctccaggaccc